SEQUENCE LISTING (4 sequences)

SEQ ID NO: 1 - DNA, 3003 bp, Homo sapiens
  CDS (536)..(2194)
cgaagtcaagacgtctggaaagaattacccagtcctggcttcgagcagcccattgaaccagagacttgaaacagccccagccaaagacttttctcccaattctgcgcttcctgggttctgctgagtcttccacaggctttttttttttttttttttttttaagacgaaaaagagattttctgttatcgggggcagaaagactgaagcacaaaaaaaaaaaaaaagaaaagaaaagaaaagaaaaaagaaaagttaatttatttttaaagcataatttttttaagaattagactgaagtgcaacggaaacataaagagaatattagtgaaattattttttaaagtggggaagaatcaaacatttaagactcccctatcctttttaaatgttgtttttaaatttcttattttttttggccggtcgtctcaaattcatctgatctcttattacctcaattttggaaactgcccgccaccgaccctccgggaccacacagacaggctgaggacgactttatgaccaagagctgaacaagatgcattgtgagaggtttctatgtatcctgagaataattggaaccacactctttggagtctctctcctccttggaatcacagctgcttatattgttggctaccagtttatccaaacggataattactatttctcttttggactgtatggtgcctttttggcatcacacctcatcatccaaagcctgtttgcctttttggagcaccgaaaaatgaaaaaatccctagaaacccccataaagttgaacaaaacagttgccctttgcatcgctgcctatcaagaagatccagactacttaaggaaatgtttgcaatctgtgaaaaggctaacctaccctgggattaaagttgtcatggtcatagatgggaactcagaagatgacctttacatgatggacatcttcagtgaagtcatgggcagagacaaatcagccacttatatctggaagaacaacttccacgaaaagggtcccggtgagacagatgagtcacataaagaaagctcgcaacacgtaacgcaattggtcttgtccaacaaaagtatctgcatcatgcaaaaatggggtggaaaaagagaagtcatgtacacagccttcagagcactgggacgaagtgtggattatgtacaggtttgtgattcagacactatgcttgacccagcctcatctgtggagatggtaaaagttttagaagaagatcccatggttggaggtgttgggggagatgtccagattttaaacaagtacgattcctggatctcattcctcagcagtgtaagatattggatggcttttaatatagaaagggcctgtcagtcttattttgggtgtgttcagtgcattagtggacctctgggaatgtacagaaactccttgttgcatgagtttgtggaagattggtacaatcaagaatttatgggcaaccaatgtagctttggtgatgacaggcatctcacgaaccgggtgctgagcctgggctatgcaacaaaatacacagctcgatctaagtgccttactgaaacacctatagagtatctcagatggctaaaccagcagacccgttggagcaagtcctacttccgagaatggctgtacaatgcaatgtggtttcacaaacatcacttgtggatgacctacgaagcgattatcactggattctttcctttctttctcattgccacagtaatccagctcttctaccggggtaaaatttggaacattctcctcttcttgttaactgtccagctagtaggtctcataaaatcatcttttgccagctgccttagaggaaatatcgtcatggtcttcatgtctctctactcagtgttatacatgtcgagtttacttcccgccaagatgtttgcaattgcaacaataaacaaagctgggtggggcacatcaggaaggaaaaccattgttgttaatttcataggactcattccagtatcagtttggtttacaatcctcctgggtggtgtgattttcaccatttataaggagtctaaaaggccattttcagaatccaaacagacagttctaattgttggaacgttgctctatgcatgctattgggtcatgcttttgacgctgtatgtagttctcatcaataagtgtggcaggcggaagaagggacaacaatatgacatggtgcttgatgtatgatcttccatgttttgacgtttgcagtcacacacaacaccttagttcctctaggggctgtacagtattgtggcatcagataatgccaccaaaggagacatatcactgctgctgggacttgaacaaagacatttatatgggtttattttcattctgccaaagtaaaacaatacatcaacaagaagaaactcagatttaacctgttatttctatgaaaatgggatgaattctttgtttatgcactttttccttactgtgcatccgcctgaaagtgttttggcctatatacctcactagccatgctttatgtgggttatcatggaagaaaaggattttggaaactcaaggaaaagttctttcaacctatacaacctaacttatggactgtttgatagatgataattttttttttttaggaaggattttctttttaactttaccaaatgaaatgccaaaggaagttttaaaggccgtggctgtgctgtatttgatataattgtactgtgtttttaaattgtgtatgccaatcttaaagacaaattttgcatattctctattttacttttctgccaaaataaacctgttcttccttttttaaaataaaataagttcttaaaaaatttatacttaaaaaatcctgcccaaaatgtgaagcttggttgactgatgttcatgatagaaagaataaaatgtttctctctctctaccttttaaaattgaatagtttatttctgtgaaagaagtatttaaactttcaatattttaactttttgtttttatttcttttagaaaaggccaatatacctatcgcg

SEQ ID NO: 2 - protein, 552 aa, Homo sapiens
MHCERFLCILRIIGTTLFGVSLLLGITAAYIVGYQFIQTDNYYFSFGLYGAFLASHLIIQSLFAFLEHRKMKKSLETPIKLNKTVALCIAAYQEDPDYLRKCLQSVKRLTYPGIKVVMVIDGNSEDDLYMMDIFSEVMGRDKSATYIWKNNFHEKGPGETDESHKESSQHVTQLVLSNKSICIMQKWGGKREVMYTAFRALGRSVDYVQVCDSDTMLDPASSVEMVKVLEEDPMVGGVGGDVQILNKYDSWISFLSSVRYWMAFNIERACQSYFGCVQCISGPLGMYRNSLLHEFVEDWYNQEFMGNQCSFGDDRHLTNRVLSLGYATKYTARSKCLTETPIEYLRWLNQQTRWSKSYFREWLYNAMWFHKHHLWMTYEAIITGFFPFFLIATVIQLFYRGKIWNILLFLLTVQLVGLIKSSFASCLRGNIVMVFMSLYSVLYMSSLLPAKMFAIATINKAGWGTSGRKTIVVNFIGLIPVSVWFTILLGGVIFTIYKESKRPFSESKQTVLIVGTLLYACYWVMLLTLYVVLINKCGRRKKGQQYDMVLDV

SEQ ID NO: 3 - DNA, 4194 bp, Mus musculus
  CDS (508)..(2166)
acatgtaagaagaaggagaagtcaaggcgtctggaaagaattacccagtcctggcttcgagcagcccattgaacgggggacttgaaccagccaaagacttcttcattctgctcttgctagactctgctgagtcttgacccggcttgtaggttgatgtgaaaagagattttgtgtcgtcggagggaaggggattggagcaaatagcaaaacagggggaaaagttaatttatctttaaagcagatataacaaagaattagaagacttaagtgcagcggaaatataaagagaatattagtgaaatttcttctcaaagaggggagaaccaagcatttaaggctcccccatctttttttttaaatgttgtttttaaatttcttattttttttggccggtcgtctcaaattcatctgatttcttattacctcaattttggaaacttccttccacgaccctccgggaccacacagacaggcggaggacgagtctatgagcaggagctgaacaagatgcattgtgagaggtttctatgtgtcctgagaataattggaactacactttttggagtgtctctcctcctcggaatcacagctgcttatattgttggctaccagtttatccaaacagataattactacttctcatttggactgtacggtgcctttttagcctcgcatctcatcatccaaagcctctttgcctttttggaacaccggaaaatgaagaagtcccttgaaaccccgattaaattgaacaaaacggtagcactctgcatcgctgcgtaccaagaggaccctgactacttacggaaatgtttgcaatctgtgaaaaggctgacctaccctgggattaaagtcgtgatggtcatcgatgggaactcagacgacgacctttacatgatggacatattcagcgaagttattggcagggacaaatcggccacgtacatctggaagaacaactttcatgaaaagggacctggtgagacagaagagtcccataaagaaagttcacaacatgtcacccaattggtcttgtctaacaaaagtatttgcatcatgcaaaaatggggtggaaagagagaagtcatgtacacagccttcagagcactggggcgaagcgtggattatgtacaggtgtgtgactcagatactatgcttgaccctgcctcatctgtggagatggtgaaggtcttagaggaagaccctatggttggaggtgttggaggagatgtccagattttaaacaagtatgattcctggatctccttcctcagcagcgtgagatactggatggcttttaatatagaaagggcctgccagtcttattttggctgtgtccagtgcataagcggtcctctgggaatgtacagaaactccttgctgcatgaatttgtggaagactggtacaatcaggaattcatgggtaaccaatgcagttttggtgacgacaggcaccttaccaacagggtgttgagtctgggctatgcaactaaatacacggctcggtccaagtgccttactgaaactcccatagaatatctgagatggctgaaccagcagacccgatggagcaagtcctacttccgagagtggctgtacaatgccatgtggtttcacaagcatcacctgtggatgacctatgaagctgttatcactggattctttcctttctttctcattgccacagtcatccagctcttctacaggggtaaaatctggaacatcctcctcttcctgttaactgtccagctagtgggtctcatcaagtcatcttttgccagctgccttagaggaaatatcgtcatggtattcatgtctctgtattcagtgttatacatgtcaagtctacttcctgccaagatgtttgcaattgcaaccataaacaaagctgggtggggcacatctggaaggaagaccattgttgttaatttcataggacttattccagtgtccgtgtggtttacaatccttctaggtggtgtaattttcaccatttataaggaatctaaaaagccattttccgaatccaaacagactgttctcatcgtgggaactttgatctatgcatgctactgggtcatgcttttgactctctatgtggttctcatcaataagtgtggcaggcggaagaagggacaacagtatgacatggtgcttgatgtatgatgatgtttgtagtcacacctggagacacacacacacacacatcacacacacacacaccttagctcctcaaggggctatacagtattgtggcaccgcaccctgccaccacaggagacatatcactgctgctgggacttgaacaaagacattcaatgggggttggtttcttttttattctgccaaagcaaattgatacatcagtgagaagaaagtccgattaaatctgacagttttaggacggtgggatgatgtcttggcttatgcacttttcccttactgtgcatctgcctgacagtgtttgttctaaatacctcacttgccatgctttgtgtgggtgatcatggaagaaaaggattctgaaaactcaagggaacgttctttcaacctacacatcctaacttatggactcttttgatagctgatgattttctttctattttttgtttttaaggaaaattgttcatctttaccaaatgaaatgccaaaggaaagttggaaagccactggctatgctgtattttgatataataattgtactgtgttttaaattttgtatccggatttttaaaaacaaaatttcacaccatagtctatattttacttctctggcaaaatacacttttgttcttttatatatatatatatatatatataataaaataggttctaaaaaaatccatactataaaaaaaaattaacctgcccaaaatgtgaaacgtggttgactgatgttcatgaaagaataaaatgtttctctctttctctacattttataattgaatagttatttctgtgaaaagaaatgtaaagtttgaatactctaacattttatttcttttagaaagggtcgagatacctgtcaacttttaggtaaaaatacatactcacatgtgtacaagagccaatcattaaagttgaggccgaaagggtagaaaagtgcaatttttgaaaatatatatttttttcaaaagggaatccatgtctttggaaaaagcaaaacaaaatgccgggctccttgtactggataaatgtgaacatccaagccagatcatctggagagatggtggcagtctttgcctaggcctagacaaaatggaaagctggtgagactttatctgtgtgattgggacaaatagaatgcaattcattaaaagttttactctgtggcctaaatgtggcagaccttctcacatgcacaatgagtgtgtttcctccagttagtgtctgggttcgccaagtcaccctgcctgttagtgatgtttacatgggcgaactgtaactgatacacaacacaattcagacctggcacatttatgttcctagtggcttctttacttctcaggaagggtatttttttttctcaattatacaggtaatctctccacttcttccatacttggccttcctaaaaatctccacaagtagaaatgatgtcaacctgtaattactattactaagaactggtaaattaaaaaaagtgacgcaccagttcctccaaacgtgtctaattcagttgtaacagggcctcagttgttaatttgacttttcacatgatttatcttggctggtgctgtgtagggctgtgagacagacactcagaacacaggaatagctgcacagaagccttgtggcgaagcaaaaagagcaccaaggttctgcttcctcactgcgcagactaccacaccacacacaactgtagcgagatactgtctaagggaaagctgcacactctaccttgtgagtacaaagaggttcgttcaagttctgaaaaactccgactctcgccgtatggagagctagtgggaaacaaacacaccctgacaataaatgaaactaaaacttgagtttgcctttttaactatttatgttctaagttaagctttgataacgttcaaatgtcaaattttttctcattcttataaaaagttgaattaattgccttgtatttattttagcaattattcaatgtatttccattataggatgtatagtataattgattgtttttgtaaataaaatatttttgat

SEQ ID NO: 4 - protein, 552 aa, Mus musculus
MHCERFLCVLRIIGTTLFGVSLLLGITAAYIVGYQFIQTDNYYFSFGLYGAFLASHLIIQSLFAFLEHRKMKKSLETPIKLNKTVALCIAAYQEDPDYLRKCLQSVKRLTYPGIKVVMVIDGNSDDDLYMMDIFSEVIGRDKSATYIWKNNFHEKGPGETEESHKESSQHVTQLVLSNKSICIMQKWGGKREVMYTAFRALGRSVDYVQVCDSDTMLDPASSVEMVKVLEEDPMVGGVGGDVQILNKYDSWISFLSSVRYWMAFNIERACQSYFGCVQCISGPLGMYRNSLLHEFVEDWYNQEFMGNQCSFGDDRHLTNRVLSLGYATKYTARSKCLTETPIEYLRWLNQQTRWSKSYFREWLYNAMWFHKHHLWMTYEAVITGFFPFFLIATVIQLFYRGKIWNILLFLLTVQLVGLIKSSFASCLRGNIVMVFMSLYSVLYMSSLLPAKMFAIATINKAGWGTSGRKTIVVNFIGLIPVSVWFTILLGGVIFTIYKESKKPFSESKQTVLIVGTLIYACYWVMLLTLYVVLINKCGRRKKGQQYDMVLDV

(19) United States
(12) Patent Application Publication
(10) Pub. No.: US 2003/0108531 A1
(43) Pub. Date: Jun. 12, 2003

(54) Genes for transfection into bony tissues

(76) Inventors: Hideshige Moriya, Chiba-shi (JP); Yuichi Wada, Chiba-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/262,526

(22) Filed: Sep. 30, 2002

(30) Foreign Application Priority Data
Nov. 30, 2001 (JP) 2001-367091

(51) Int. Cl. A61K 48/00; C12N 15/87; C12N 5/08; C12M 1/00

(52) U.S. Cl. 424/93.21; 514/44; 435/366; 435/459; 435/285.3

(57) ABSTRACT

A gene which is used for transfection into a bony tissue with a gene gun. The gene can well repair a cartilage defective portion to a nearly normal state by transfecting it in the bony tissue for transplantation in the cartilage defective portion without affecting the bony tissue.

### BACKGROUND OF THE INVENTION

1. Field of the Invention

The present invention relates to a gene and a carrier which are used for transfection into a bony tissue with a gene gun, a carrier which carries the gene, and the like.

2. Description of the Related Art

As a method for repairing an articular cartilage, various methods such as drilling, curettage, osteochondral transplantation, periosteal transplantation, perichondreal transplantation, synovial transplantation, chondrocostal transplantation and culture chondrocyte transplantation have been reported (Fraenkel SR et al., A Comparison of Abrasion Burr Arthroplasty and Subchondral Drilling in the Treatment of Full Thickness Cartilage Lesions in the Rabbit, The Transactions of the Orthopaedic Research Society, 19:483, 1994, Salter R B, The Biological Effect of Continuous Passive Motion on the Healing of Full Thickness Defects in Articular Cartilage, An Experimental Investigation in the Rabbit, JBJS, 62A: 1232-1251, December 1980, Scradge H. et al., Perichondral Resurfacing Arthroplasty in the Hand, J. of Hand Surg., 9A: 880-886, 1984, Outerbridge H K et al., The Use of a Lateral Patellar Autogenous Graft for the Repair of Large Osteochondral Defects in the Knee, JBJS 77A: 65-72, January 1995, Brittberg M. et al., Treatment of deep cartilage defects in the knee with autologous chondrocyte transplantation, New Engl. J. Med., 331: 889-895, 1994).

In recent years, a method for transfecting genes into articular chondrocytes in vivo or ex vivo has been developed for cartilage repair. In the in vivo method, vectors are directly injected into a joint, and in the ex vivo method, chondrocytes having genes transfected therein are transplanted into a cartilage.

In the transfection of genes into chondrocytes, the use of viral vectors or liposomes has been reported. However, a method which is even easier, quicker and higher in safety has been in demand.

Meanwhile, in a method for transfecting genes with a gene gun, desired foreign genes are transfected into target tissues or cells along with metallic particles. In this method, genes can be transfected easily and quickly. However, since metallic particles and the like are directly shot into tissues or cells, cells might be damaged or functions of tissues might be lost. For this reason, the use thereof in the transplantation field for cartilage repair has not been reported at all.

### SUMMARY OF THE INVENTION

Under these circumstances, the invention has been made, and it aims to provide a gene and a carrier which are used for transfection into a bony tissue with a gene gun, a carrier which carries the gene and the like.

For solving the problems, the present inventors have assiduously conducted investigations, and have consequently found that the physical transfection of genes into a bony tissue with a gene gun does not have any adverse effect on the bony tissue itself, nor does it substantially impair functions of the bony tissue itself, that when this bony tissue is transplanted into a cartilage defective portion, it is integrated therein as in a normal periosteum to form a cartilage, that genes transfected into a periosteum are properly expressed at an appropriate time and the gene expression is reduced when it is unnecessary, that this gene expression is substantially free from disorder in the bony tissue, and that when specific genes are transfected into a bony tissue and the resulting bony tissue is used for transplantation into a cartridge defective portion, this defective portion can be repaired well to a nearly normal state. These findings have led to the completion of the invention.

That is, the invention provides a gene which is used for transfection into a bony tissue with a gene gun (hereinafter referred to as a “gene of the invention”).

The gene of the invention is preferably DNA encoding an enzyme protein, more preferably DNA encoding a hyaluronic acid synthetase, further preferably DNA encoding hyaluronic acid synthetase 2. Especially, the DNA encoding hyaluronic acid synthetase 2 is preferably selected from the following (a) to (c).

(a) DNA encoding a protein comprising an amino acid sequence represented by SEQ ID NO: 2.

(b) DNA encoding a protein comprising an amino acid sequence represented by SEQ ID NO: 2 in which one or several amino acids are deleted, substituted, inserted or transposed, the protein having a hyaluronic acid synthetase activity.

(c) DNA which is hybridizable with the DNA described in (a) or DNA complementary to the DNA described in (a), or DNA having a part of nucleotide sequences of these DNAs under stringent conditions.

Of these, DNA comprising a nucleotide sequence represented by nucleotide Nos. 536 to 2191 in SEQ ID NO: 1 is extremely preferable.

These genes are preferably carried by a vector, and this vector is preferably an expression vector.

The bony tissue in which the gene of the invention is transfected is preferably a periosteal tissue or a cartilaginous tissue, and these tissues are preferably to be grafted into a defective portion of an articular cartilage.

Further, the invention provides a carrier which is used for transfecting a gene into a bony tissue with a gene gun (hereinafter referred to as a “carrier of the invention”). Among others, a carrier which carries the gene of the invention is preferable.

Still further, the invention provides a kit comprising the gene and the carrier of the invention as constituents (hereinafter referred to as a “kit of the invention”).

Furthermore, the invention provides a bony tissue transfected with the gene of the invention (hereinafter referred to as a “tissue of the invention”).

Moreover, the invention provides a gene gun which is used for transfecting the gene of the invention into a bony tissue (hereinafter referred to as a “gene gun of the invention”).

### BRIEF DESCRIPTION OF THE DRAWINGS

FIG. 1 shows a hematoxylin-eosin (HE) stained image of a LacZ transfection group in the 2nd week,

FIG. 2 shows a toluidine blue (TB) stained image of a LacZ transfection group in the 2nd week,

FIG. 3 shows an HE stained image of a LacZ transfection group in the 4th week,

FIG. 4 shows a TB stained image of a LacZ transfection group in the 4th week,

FIG. 5 shows histological evaluation in the 2nd week (2W) and the 4th week (4W) in which the ordinate represents scores according to the Wakitani method,

FIG. 6 shows an Xgal stained image in the 2nd week,

FIG. 7 shows an Xgal stained image in the 4th week,

FIG. 8 shows an Xgal stained image in the 12th week,

FIG. 9 shows effects of gene transfection into the 2nd week (2W), the 4th week (4W) and the 12th week (12W),

FIG. 10 shows an HE stained image of an HAS 2 transfection group in the 2nd week,

FIG. 11 shows a TB stained image of an HAS 2 transfection group in the 2nd week,

FIG. 12 shows histological evaluation in the 2nd week in which Cont is short for control, and A: cell morphology, B: intracellular matrix staining, C: surface regularity, D: thickness of a cartilage, and E: integration of a tissue,

FIG. 13 shows an HE stained image of an HAS 2 transfection group in the 4th week,

FIG. 14 is a TB stained image of an HAS 2 transfection group in the 4th week,

FIG. 15 shows histological evaluation in the 4th week in which symbols are the same as in FIG. 12,

FIG. 16 shows histological evaluation in the 2nd week (2W) and the 4th week (4W), and

FIG. 17 shows expression of type I collagen and type II collagen in the 4th week.

### DETAILED DESCRIPTION OF THE INVENTION

<1> Gene of the Invention

The gene of the invention is characterized in that it is used for transfection into a bony tissue with a gene gun. Preferably, the gene of the invention is used as an active ingredient of a composition for gene therapy for transfecting the same into the bony tissue with the gene gun.

The “gene gun” referred to in the invention is a unit in which a foreign gene is carried on a carrier and the carrier is to be shot into cells or tissues to transfect the foreign gene into cells or tissues. Any guns are encompassed in the concept of the gene gun of the invention so long as they have such a performance. For example, a gene gun and a particle gun are included in the gene gun of the invention.

In the invention, the “bony tissue” is a concept including a periosteal tissue, a perichondreal tissue, a synovial tissue and a cartilaginous tissue.

The gene of the invention is not particularly limited so long as the desired function is exhibited by transfection into the bony tissue with the gene gun. Specifically, DNA encoding a protein is preferable, and DNA encoding a protein having any physiological activity is preferable. The protein having any physiological activity includes an enzyme protein, an induction factor and a growth factor. A protein that expedites formation or regeneration of a tissue or participates in repair of a tissue is preferable. Examples of such an induction factor or growth factor include bone morphogenetic proteins (BMPs) and transforming growth factor β (TGF-β).

As the gene of the invention, DNA encoding an enzyme protein is especially preferable. As DNA encoding the enzyme protein, DNA encoding a hyaluronic acid synthetase (hereinafter referred to also as “HAS”) is preferable. For example, as HAS of mammals, HAS 1 (Biol. Biochem. Res. Commun., 222 (1996), p. 816), HAS 2 and HAS 3 (WO 98/00551) are known (Genomics, 41(3), p. 493-497 (1997)), and DNAs encoding the same can also be used. The dose of HAS 2 expressed in the articular cartilage is the highest, and the dose of HAS 3 expressed therein is the second highest. Therefore, as the gene of the invention, DNA encoding HAS 2 or HAS 3 is preferable, and DNA encoding HAS 2 is more preferable.

With respect to DNA (cDNA) encoding HAS 2, DNA derived from humans can be prepared by the method described in J. Biol. Chem., vol. 271 (1996), No. 38, pp. 22945-22948, and DNA derived from mice by the method described in J. Biol. Chem., vol. 271 (1996), No. 38, pp. 23400-23406 respectively.

The origin of the protein encoded by the gene of the invention (animal species) is not particularly limited either. The protein is preferably derived from the same animal as the bony tissue in which the gene of the invention is to be transfected. The origin of the bony tissue in which the gene of the invention is transfected (animal species) is preferably the same as an animal (host) in which the bony tissue is to be transplanted, and the same individual is more preferable. The gene of the invention is preferably used for transplantation in, for example, a cartilage defective portion of vertebrates, preferably mammals, more preferably humans. For this reason, the origin of the bony tissue in which the invention gene is transfected is vertebrates, preferably mammals, more preferably humans.

Accordingly, the origin of the protein encoded by the gene of the invention (animal species) is vertebrates, preferably mammals, more preferably humans. When DNA encoding HAS 2 is used as the gene of the invention, DNA encoding HAS 2 derived from humans is preferable. Specifically, any of the following (a) to (c) is preferable.

(a) DNA encoding a protein comprising an amino acid sequence represented by SEQ ID NO: 2.

By the way, it can easily be understood by those skilled in the art that as DNA encoding the protein, there are DNAs having various nucleotide sequences owing to degeneracy of the genetic code.

Of these, DNA comprising a nucleotide sequence represented by Nucleotide Nos. 536 to 2191 in SEQ ID NO: 1 is specifically preferable.

(b) DNA encoding a protein comprising an amino acid sequence represented by SEQ ID NO: 2 in which one or several amino acids are deleted, substituted, inserted or transposed, the protein having a hyaluronic acid synthetase activity.

(c) DNA which is hybridizable with the DNA described in (a) or DNA complementary to the DNA described in (a), or DNA having a part of nucleotide sequences of these DNAs under stringent conditions.

When DNA encoding HAS 2 derived from mice is used as the gene of the invention, any of the following (a) to (c) is preferable.

(a) DNA encoding a protein comprising an amino acid sequence represented by SEQ ID NO: 4.

Especially, DNA comprising a nucleotide sequence represented by Nucleotide Nos. 508 to 2163 in SEQ ID NO: 3 is specifically preferable.

(b) DNA encoding a protein comprising an amino acid sequence represented by SEQ ID NO: 4 in which one or several amino acids are deleted, substituted, inserted or transposed, the protein having a hyaluronic acid synthetase activity.

(c) DNA which is hybridizable with the DNA described in (a) or DNA complementary to the DNA described in (a), or DNA having a part of nucleotide sequences of these DNAs under stringent conditions.

DNAs (b) and (c) include DNAs structurally different from DNA (a) (DNA encoding natural HAS 2) but encoding proteins having the same function as HAS 2.

That is, it is known that although naturally occurring proteins might undergo mutation such as substitution, deletion, insertion or transposition of amino acids in the amino acid sequence owing to polymorphism or mutation of DNAs encoding the proteins or a modification reaction in cells of proteins after synthesis or during purification of the proteins, some of such proteins show substantially the same physiological and biological activities as proteins free from mutation. Thus, DNAs encoding proteins which are somewhat different structurally but not quite different functionally from natural HAS 2 are also included in DNA encoding HAS 2.

Further, since DNAs cause substitution, deletion, insertion or transposition of amino acids in an amino acid sequence, it is also possible to artificially substitute, delete, insert or transpose nucleotides in nucleotide sequences of DNAs.

Such substitution, deletion, insertion or transposition of nucleotides in the nucleotide sequence of DNA can be introduced by synthesizing a sequence including mutation site(s) and having restriction enzyme cleavage ends on both termini and replacing a corresponding portion of a nucleotide sequence of unmutated DNA therewith. Moreover, substitution, deletion, insertion or transposition of nucleotides can also be introduced in DNA by the site specific mutation method (Kremer, W. and Frits, H. J., Meth. In Enzymol., 154, 350 (1987); Kunkel, T. A. et al., Meth. In Enzymol., 154, 367 (1987)).

In the invention, “several amino acids” indicate the number of amino acids which may cause mutation that does not lose the HAS 2 activity. For example, in case of a protein comprising 600 amino acid residues, the number is from 2 to 30, preferably from 2 to 15, more preferably from 2 to 8.

The gene of the invention is not necessarily a gene consisting of a single gene, and it may contain plural types of genes in a molecule. For example, it may be DNA containing a region encoding HAS 2 and a region encoding BMP (or TGF-β) in a molecule. In this case, two types of genes are contained in a molecule. Likewise, it may be DNA further containing a region encoding TGF-β (or BMP). In this case, three types of genes are contained in a molecule. Such genes of the invention can be prepared by cleaving and ligating plural DNAs by a known genetic engineering method.

The “hyaluronic acid synthetase activity” can be detected by, for example, the method described in J. Biol. Chem., 271(17), pp. 9875-9878 (1996). Deletion, substitution, insertion or transposition of amino acids having the hyaluronic acid synthetase activity can easily be selected by this method.

In the present invention, the “stringent conditions” indicate conditions under which a specific hybrid is formed and a nonspecific hybrid is not formed (refer to Sambrook, J. et al., Molecular Cloning A Laboratory Manual, second edition, Cold Spring Harbor Laboratory Press (1989)). As the “stringent conditions”, conditions are specifically proposed in which hybridization is performed in a solution containing 50% formamide, 4×SSC, 50 mM HEPES (pH 7.0), 10× Denhardt's solution and 100 μg/ml salmon sperm DNA at 42° C. and washing is performed in a solution containing 2×SSC and 0.1% SDS at room temperature and in a solution containing 0.1×SSC and 0.1% SDS at 50° C.

A process for producing the gene of the invention is not particularly limited. It can be produced by properly obtaining a desired gene for transfection into a bony tissue. For example, the gene of the invention can be produced by extracting or amplifying a desired gene which is to be transfected into a bony tissue from natural sources. Further, the gene of the invention can also be obtained by genetic engineering techniques such that a desired gene is obtained from cells transformed with the gene once cloned. Moreover, it can also be produced by chemical synthesis. The thus-produced desired genes can all be used as the gene of the invention.

It is preferable that the gene of the invention is carried by a vector for stably maintaining its molecule and properly exhibiting its function in a bony tissue transfected with the gene. Specifically, the gene of the invention carried by a vector can be prepared by inserting a desired gene to be transfected into a bony tissue into a known vector.

The vector for inserting the desired gene to be transfected into a bony tissue can properly be selected depending on a gene function to be exhibited or an origin of a bony tissue for transfection (animal species). An expression vector containing an appropriate expression control sequence such as a promoter is preferable. As the expression vector, a plasmid vector can be proposed, and it can properly be selected by those skilled in the art according to a desired gene to be expressed. For example, when a desired gene to be transfected into a bony tissue is DNA encoding human HAS 2, expression vectors for mammal cells, such as pcDNA3, pGIR201 (Kitagawa, H., and Paulson, J. C. (1994) J. Biol. Chem. 269, 1394-1401), pEF-BOS (Mizushima, S., and Nagata, S. (1990) Nucleic Acid Res. 18, 5322), pCXN2 (Niwa, H., Yamanura, K. and Miyazaki, J. (1991) Gene 108, 193-200), pCMV-2 (Eastman Kocak), pCEV18, pME18S (Maruyama et al., Med. Immunol., 20, 27 (1990)) and pSVL (Pharmacia Biotech) can be used.

In case of using any of these vectors, for enabling ligation of the desired gene to be transfected into the bony tissue with the vector, both are treated with restriction enzyme(s), and blunting or ligation with cohesive ends is conducted as required. Then, the desired gene and the vector can be ligated.

The gene of the invention is characterized in that it is used for transfection into the bony tissue with the gene gun. The bony tissue into which the gene is transfected is preferably a periosteal tissue or a cartilaginous tissue. These tissues are preferably grafted into a defective portion of a cartilage, more preferably into a defective portion of an articular cartilage. The bony tissue in which the gene of the invention is transfected is, when used in transplantation, preferably collected from an individual which is the same as an individual (host) for transplantation.

The conditions for transfection with the gene gun can properly be determined by those skilled in the art depending on the type of the gene gun to be used, the type or the size of the bony tissue into which the gene is transfected, and they are not particularly limited. For example, in case of using a high-pressure helium gas, the pressure is set at, preferably from 100 to 600 psi, more preferably from 200 to 400 psi, further preferably 200 psi. Incidentally, 1 psi is 6,890 Pa.

The gene of the invention may comprise one type of a desired gene alone or it may be a mixture of plural types of desired genes. For example, it may be a mixture of DNA encoding HAS 2 and DNA encoding BMP (or TGF-β). In this case, the gene of the invention comprises two types of genes. It may further contain DNA encoding TGF-β (or BMP). In this case, the gene of the invention comprises three types of genes.

The gene of the invention can be stored or distributed in various states in which it is dissolved in an appropriate solvent (liquid state), frozen or dried. Further, for maintaining a stability of the gene of the invention, it may contain pharmaceutically acceptable ingredients as required. Still further, it may contain pharmacologically active ingredients in addition to the gene of the invention. That is, the gene of the invention may be substantially free from other ingredients or a mixture with other ingredients (composition).

The gene of the invention is used for production of the “carrier of the invention which carries the gene of the invention” or a “composition for gene therapy containing the gene of the invention and being introduced into the bony tissue with the gene gun”. The carrier or the composition is then introduced into the bony tissue with the gene gun to transfect the gene of the invention into the bony tissue. A process for producing the “carrier of the invention which carries the gene of the invention” is described below.

<2> Carrier of the Invention

The carrier of the invention is characterized in that it is used for transfecting the gene into the bony tissue with the gene gun. The carrier of the invention is preferably a carrier used to produce the composition for gene therapy in which the gene is transfected into the bony tissue with the gene gun.

The form, the material and the size of the carrier of the invention are not particularly limited so long as the carrier is one ordinarily used to introduce a gene with a gene gun, and a commercial carrier is available. For example, as the form of the carrier, a nearly spherical carrier can be proposed. As the material of the carrier, a material which less affects cells or tissues is preferable, and gold can be proposed. The carrier can have such a size as to identify it as a particle. Specifically, particles having a diameter of from 1.0 to 2.0 μm can be proposed. Among others, particles having a diameter of 1.0 μm are preferable. It is most preferable that the carrier of the invention is nearly spherical gold particles having a diameter of 1.0 μm.

It is advisable that an ingredient for enhancing a retention efficiency or a stability of the gene of the invention is coated on the surface of the carrier of the invention. For example, since gene (DNA) is acid, a basic material is coated, whereby a retention efficiency or a stability of the gene of the invention can be enhanced. Preferable examples of the material include spermidine spermine and polylysine. For example, when gold particles are coated with these materials, it is advisable that gold particles are contacted with these materials. Thus, the product obtained by coating the other ingredient on the surface of the carrier of the invention is also included in the concept of the carrier of the invention.

The carrier of the invention is characterized in that it is used for transfecting the gene into the bony tissue with the gene gun. The preferable bony tissue, the conditions for transfecting the gene with the gene gun and the like are the same as described in <1> gene of the invention.

The carrier of the invention may comprise one type of a carrier or be a mixture of plural types of carriers. For example, it may be a mixture of carriers different in form, material or size. In this case, the carrier of the invention contains plural types of carriers.

The carrier of the invention can be stored or distributed in various states in which it is suspended in an appropriate solvent (slurry), or dried. Further, the carrier of the invention may contain ingredients for preventing denaturation thereof as required. That is, the carrier of the invention may be substantially free from other ingredients or a mixture (composition) with other ingredients.

The carrier of the invention is used for production of the “carrier of the invention which carries the gene of the invention”. Then, the carrier is introduced into the bony tissue with the gene gun to introduce the gene of the invention carried by the carrier of the invention into the bony tissue.

Further, the “carrier of the invention which carries the gene of the invention” can be produced by affixing the gene of the invention on the carrier of the invention. The method for affixing is not particularly limited, and it can properly be selected depending on the form, the material and the size of the carrier of the invention. For example, when gold particles coated with spermidine are used as the carrier of the invention, the surfaces thereof are coated with the genes of the invention physically or chemically, whereby the genes of the invention can be carried on the carriers of the invention. In this case, the coating can be conducted by contacting the gold particles coated with spermidine with the genes of the invention. The preferable coating method will be described later in Examples.

Since the resulting “carrier of the invention which carries the gene of the invention” is an example of the carrier of the invention, it can be stored and distributed in various states as described earlier, and may contain desired ingredients as required. That is, the “carrier of the invention which carries the gene of the invention” may be substantially free from other ingredients or a mixture (composition) with other ingredients. A preferable embodiment of the composition for gene therapy is such an invention carrier.

The “carrier of the invention which carries the gene of the invention” is characterized in that it is used for transfecting the gene into the bony tissue with the gene gun.

<3> Kit of the Invention

The kit of the invention comprises the gene of the invention and the carrier of the invention as constituents. The kit of the invention is characterized in that it is used for transfecting the gene into the bony tissue with the gene gun.

The “gene of the invention” and the “carrier of the invention” which are the constituents of the kit of the invention are as described in <1> gene of the invention and <2> carrier of the invention.

In the kit of the invention, it is possible that the “gene of the invention” and the “carrier of the invention” are retained in independent containers and combined to produce the kit of the invention which is distributed. When the gene is transfected into the bony tissue with the gene gun, the “carrier of the invention which carries the gene of the invention” is produced using the “gene of the invention ” and the “carrier of the invention” as the constituents of the kit, and subjected to the gene transfection. A process for producing the “carrier of the invention which carries the gene of the invention” is as described in <2> carrier of the Invention.

The kit of the invention may contain other constituents so long as it comprises the “gene of the invention” and the “carrier of the invention” as constituents. For example, besides the “gene of the invention gene” and the “carrier of the invention carrier”, it may contain an ingredient to be coated on the carrier of the invention for enhancing a retention efficiency or a stability of the gene of the invention, a solution for suspending the carrier of the invention which carries the gene of the invention and a tube (cartridge of the gene gun) for temporarily holding the carrier of the invention which carries the gene of the invention.

The ingredient to be coated on the carrier of the invention for enhancing a retention efficiency or a stability of the gene of the invention is as described in <2> carrier of the invention carrier. The solution for suspending the carrier of the invention which carries the gene of the invention is not particularly limited. For example, an ethanol solution of polyvinyl pyrrolidone (PVP) is proposed. Further, as the tube (cartridge of the gene gun) for temporarily holding the carrier of the invention which carries the gene of the invention, a commercial tube for a gene gun can be used.

<4> Tissue of the Invention

The invention tissue is a bony tissue in which the gene of the invention is transfected. The invention tissue is characterized in that the gene is transfected therein with the gene gun. It is preferably a bony tissue in which the gene for gene therapy is transfected with the gene gun.

The gene of the invention and the preferable invention bony tissue are as described in <1> gene of the invention.

The invention tissue can be produced by introducing the “carrier of the invention which carries the gene of the invention” into the bony tissue with the gene gun. The conditions for introduction with the gene gun are as described in <1> gene of the invention. The “carrier of the invention carrier which carries the gene of the invention” used in the gene transfection with the gene gun is also as described in <2> carrier of the invention.

The invention tissue can be used for transplantation into a defective portion of a cartilage, especially a defective portion of an articular cartilage. The invention tissue may be produced in this transplantation or previously produced and stored in a tissue culture solution.

<5> Gene Gun of the Invention

The gene gun of the invention is characterized in that it is used for transfecting the gene of the invention into the bony tissue.

The gene of the invention is as described in <1> gene of the invention.

The gene gun of the invention can be produced, for shooting the carrier for gene transfection which carries the gene of the invention into the bony tissue to introduce the gene of the invention, as a unit provided with a tube for accelerating a gas such as helium and a mechanism for storing a carrier for gene transfection which carries a gene, and having a size suited for introduction in a bony tissue. A commercial gene gun can be used as such.

### EXAMPLES

The invention is illustrated specifically below by referring to Examples. However, the technical scope of the invention is not limited by these Examples.

1. Preparation of a Gene for Transfection

(1) LacZ Gene

β-Galactosidase expression vector pCMVβ (7.2 kb; Clontech) was used.

(2) HAS 2 Gene

DNA encoding HAS 2 (represented by Nucleotide Nos. 508 to 2163 in SEQ ID NO: 3) was procured from Prof. Koji Kimata, Institute for Molecular Science of Medicine, Aichi Medical University. As DNA encoding HAS 2, DNA integrated in vector pcDNA3 was used.

2. Preparation of a Carrier Which Carries a Gene

A carrier was prepared according to the method described in the manual of Helios Gene Gun System of Nippon Bio-Rad Laboratories. That is, 25 mg of nearly spherical gold particles (diameter 1 μm, Nippon Bio-Rad Laboratories) washed was first added to 100 μl of 0.05 M spermidine (Sigma), and vigorously stirred. Then, 100 μg of the genes for transfection was added, and the mixture was re-stirred vigorously. The suspension was mixed with 100 μl of a 1M CaCl₂ solution. The mixture was then vigorously stirred, and 10 minutes later, centrifuged at 10,000 rpm. After the centrifugation, the precipitate was washed with ethanol, and dried. Gold particles coated with DNAs were suspended in 3 ml of a polyvinyl pyrrolidone solution (solution dissolved in 99.5% ethanol at a concentration of 0.02 mg/ml, Nippon Bio-Rad Laboratories). The suspension of the gold particles was charged into a Tefzel tube (Nippon Bio-Rad Laboratories), and dried in a nitrogen stream (0.3 ml/min) for 15 minutes while being rotated. Then, the tube was cut to a length of 1.2 cm (cartridge), and stored along with silica gel at −20° C. until used. Approximately 4 mg of the genes (DNAs) for transfection was coated on the surfaces of 1 mg of the gold particles.

3. Experimental Animal

New Zealand white rabbits (NZW rabbits, male) each having an average weight of 3.20 kg (from 2.90 to 3.50 kg) were used. Anesthesia in operation was conducted by injecting 40 mg/kg.weight of ketamine and 6 mg/kg.weight of xyladine subcutaneously or intramuscularly.

4. Experiments of LacZ Gene Transfection and Transplantation

Knees of 12 NZW rabbits (male, 3.2 kg) was grouped as follows.

1) control group 10 knees

2) LacZ transfection group (group with LacZ gene transfected) 14 knees

(1) Production of a Cartilage Defect

By intrusion into a joint upon cutting an inside patellar skin, a cartilage piece was collected from a medial epicondyle of femur constituting a patellar femoral joint to produce a cartilage defect of 5 mm×4 mm×2 mm (depth).

(2) Collection and Treatment of a Periosteum Used for Transplantation

A periosteum (7 mm×7 mm) for transplantation was collected from an inner proximal tibia of each individual (total of 24 periostea).

With respect to 14 periostea for transplantation in the LacZ transfection group, the gold particles (coated with pCMVβ) prepared in 2 above were shot into osteogenetic layers of the periostea with a high-pressure helium gas (200 psi: ipsi=6,890 Pa) using Helios Gene Gun System. Consequently, the gold particles penetrated the cell membranes of the cells in the periostea for transplantation in the LacZ transfection group to introduce the LacZ genes (LacZ group).

The 10 periostea for transplantation in the control group were not treated at all.

(3) Transplantation of a Periosteum Into a Cartilage Defective Portion

The periosteum collected and treated in (2) was put on the joint surface in the corresponding cartilage defective portion (produced in (1)) of each group with the osteogenetic layer laid down, and sutured with a 5-0 polygalactin yarn (VICRYL Coat, Ethicon) through holes 0.7 mm in diameter which were formed in four corners of the defective portion with a drill. Each of the individuals after operation was caused to freely move in a case and to properly drink water and feed. In the 2nd week (n=5), the 4th week (n=5) and the 12th week (n=2) after operation, the individuals were subjected to euthanasia using CO₂. The overall knee joint was extracted, and visual observation and histological evaluation were performed.

(4) Observation and Evaluation

The visual observation was performed by observing an appearance and a color of a knee joint, a thickness of a newly formed tissue, an extent of a surface smoothness, arthritis or erosion around the joint (femur, patella and tibia) through visual examination, synovitis, condition of lunula and contracture of any joint.

The histological evaluation of the cartilage defective portion (periosteum transplantation portion) was performed by conducting hematoxylin-eosin (HE) staining and toluidine blue (TB) staining and then scoring the following items according to the Wakitani method (Wakitani S. et al.: Repair of rabbit articular surfaces with allograft chondrocytes embedded in collagen gel, J. Bone Joint Surg., 71-B: 74-80, 1989).

| (a) Cell morphology | |
|---|---|
| hyaline cartilage | 0 |
| mostly hyaline cartilage | 1 |
| mostly fibrocartilage | 2 |
| mostly non-cartilage | 3 |
| non-cartilage only | 4 |
| (b) Intracellular matrix staining(metachromasia) | |
| same metachromasia as in a host cell | 0 |
| Metachromasia is slightly reduced. | 1 |
| Metachromasia is markedly decreased. | 2 |
| no metachromasic stain | 3 |
| (c) Surface regularity | |
| At least ¾ of a defective region is smooth. | 0 |
| At least ½ and less than ¾ of a defective region | 1 |
| is smooth. | |
| At least ¼ and less than ½ of a defective region | 2 |
| is smooth. | |
| Less than ¼ of a defective region is smooth. | 3 |
| (d) Thickness of a cartilage | |
| The thickness is at least ⅔ the thickness of the | 0 |
| surrounding cartilage. | |
| The thickness is at least ⅓ and less than ⅔ the | 1 |
| thickness of the surrounding cartilage. | |
| The thickness is less than ⅓ the thickness of the | 2 |
| surrounding cartilage. | |
| (e) Integration of donor with host adjacent cartilage | |
| Both edges integrated. | 0 |
| One edge integrated. | 1 |
| Neither edge integrated. | 2 |

A total of scores (a) to (e) was used as an overall score. The highest score was 14. The lower the score, the better the joint repair.

As a result of the visual observation, in the 2nd week, the periosteum-like fibrous tissue was observed in the cartilage defective portion somewhat integrated in the surrounding cartilage in both the LacZ transfection group and the control group. In the 4th week, the newly formed tissue became white, and looked like a cartilage. In the 12th week, the tissue was somewhat glossy, and looked like a cartilage. However, it was somewhat whitish in comparison with the surrounding normal cartilage. No clear difference was found between the two groups. In both groups, slight erosion was observed in middle and side edges on a joint front of a femur. In some samples, slight arthritic variation was observed in the cartilage of the patella. However, it was not observed in most of the samples. Symptoms of synovitis, lesion of menisci and joint contracture were not observed in the two groups.

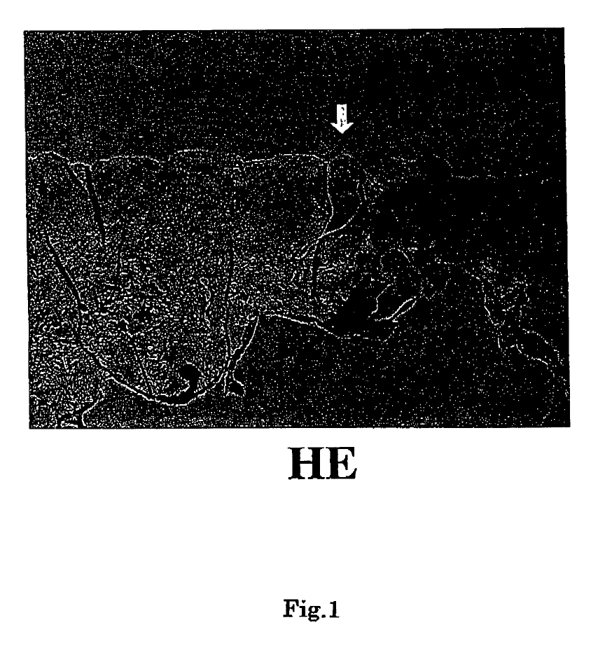

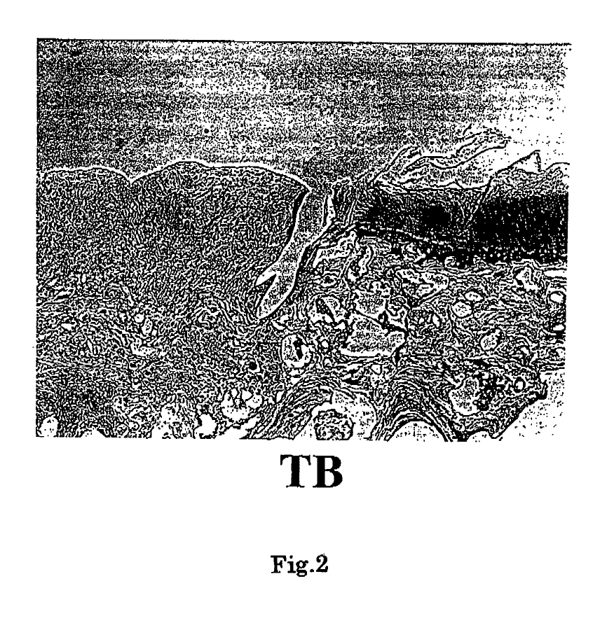

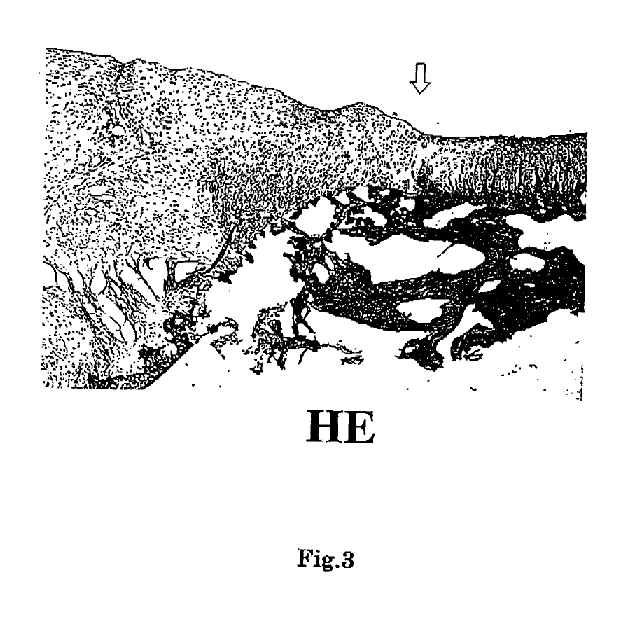

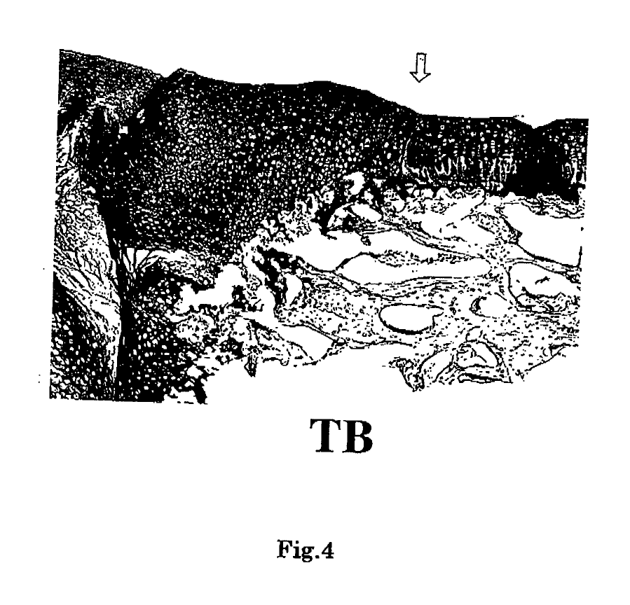

The typical HE stained image and TB stained image of the LacZ transfection group in the 2nd week are shown in FIGS. 1 and 2 respectively, and the typical HE stained image and TB stained image of the LacZ transfection group in the 4th week are shown in FIGS. 3 and 4 respectively.

FIGS. 1 and 2 revealed that in the 2nd week, the edge of the transplanted periosteum was not integrated in the surrounding normal cartilage, and that the repaired tissue comprised the fibrous tissue not showing metachromasia by the TB staining.

FIGS. 3 and 4 revealed that in the 4th week, the fibrous tissue was replaced with the undifferentiated cartilaginous tissue having a metachromatic matrix and integrated well in the surrounding normal cartilage. Circular or elliptical chondrocyte-like cells having voids were observed in the deeper layer of the tissue.

In the 12th week, it was observed that the repaired tissue was made almost completely of a hyaline cartilage stained well with TB.

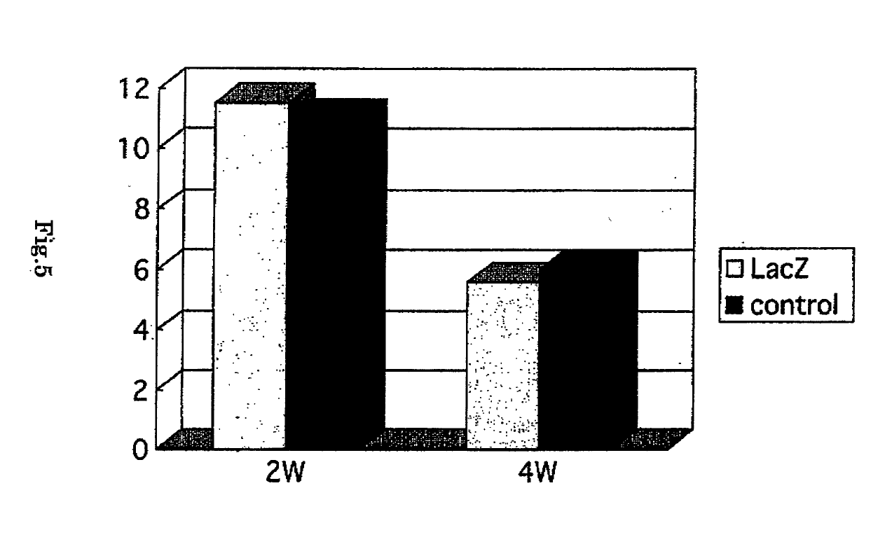

The histological evaluation (average of overall scores in each group) of the control group and the LacZ transfection group in the 2nd and 4th weeks is shown in FIG. 5.

In FIG. 5, no significant histological difference was observed between the control group and the LacZ transfection group. Moreover, no significant difference was observed either therebetween in the items, cell form, intracellular matrix staining, surface condition, thickness of a cartilage and integration in an adjacent cartilage portion.

Accordingly, it was shown that even though the gene transfection was physically conducted in the periosteum with the gene gun, the function of the periosteum itself was not substantially impaired. Thus, it was identified that when this periosteum was transplanted in the cartilage defective portion, it was, like the normal periosteum, integrated to form a cartilage (to repair the tissue).

Next, in order to examine whether or not the gene (LacZ gene) transfected into the periosteum exhibited the function in the periosteal tissue (whether or not β-galactosidase was expressed with the LacZ gene), the LacZ transfection group was subjected to Xgal (5-bromo-4-chloro-3-indolyl-β-D-galactoside) staining in the 2nd, 4th and 12th weeks.

In the Xgal staining, the newly formed tissue was cut out in a block state along with the surrounding normal cartilage and bones under the cartilage, and fixed at 4° C. for 30 minutes using 1% formaldehyde and 0.2% glutaraldehyde. The sample was washed with PBS, and then treated at 37° C. for 6 hours using a 0.1 M sodium phosphate buffer solution (pH 7.5), 10 mM KCl, 3 mM K₄Fe(CN)₆, 3 mM K₃Fe(CN)₆, 1 mM MgCl₂, 0.1% Triton X-100 and 1 mM 5-bromo-4-chloro-3-indolyl-β-D-galactopyranoside (Xgal). The reaction was terminated by washing with PBS containing 1 mM EDTA. Subsequently, the sample was fixed with PBS containing 4% paraformaldehyde, decalcified at 4° C. for 3 days using a 0.15 M NaCl solution containing 20% EDTA, and frozen in OCT Compound (Miles Scientific). Thereafter, a continuous piece having a thickness of 5 μm was formed, and stained with hematoxylin.

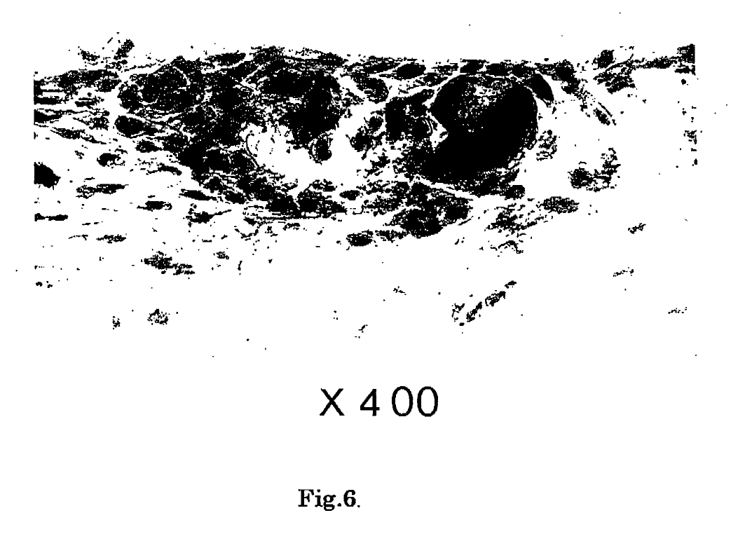

The typical Xgal stained images in the 2nd, 4th and 12th weeks are shown in FIGS. 6 to 8 respectively.

FIG. 6 revealed that in the LacZ transfection group in the 2nd week, the gold particles and the cells stained blue by the Xgal staining were observed mainly on the surface layer of the transplanted periosteum, whereas in the control group, these were not observed. The site containing many gold particles was stained more strongly. This observation suggested that the LacZ genes were transfected in cells of the osteogenetic layer with the gene gun to express the LacZ genes.

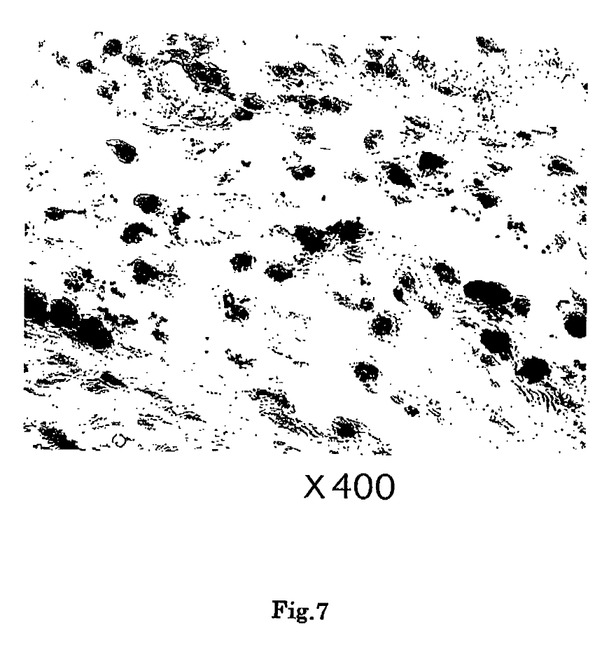

FIG. 7 revealed that the LacZ gene positive cells were still observed in the 4th week but the number thereof was smaller than in the 2nd week. In some samples, the growth of the periosteal tissue was observed around the LacZ positive cells of the osteogenetic layer.

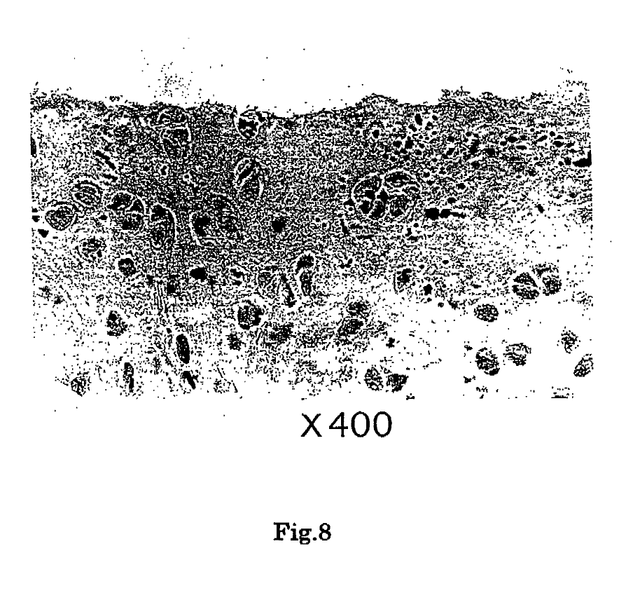

FIG. 8 revealed that in the 12th week, the Xgal stained cells of the repaired tissue were not observed and only the gold particles remained on the surface layer.

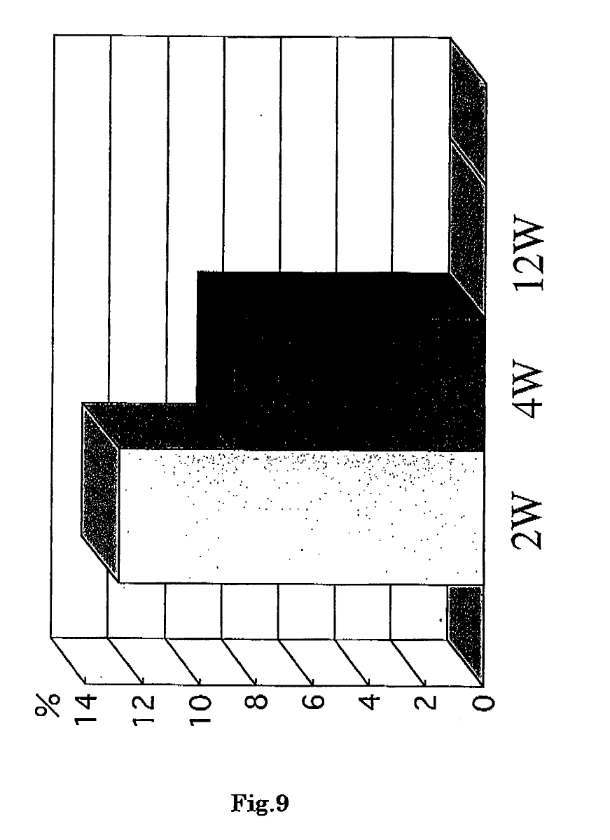

Most of the LacZ positive cells were observed within the depth of 100 μm, and the LacZ positive cells were slightly observed in the deeper site. The positive cells were mainly observed in the vicinity of the center of the transplanted periosteal piece. In the central portion (300 μm×300 μm×100 μm (depth)) of the sample, the total number of cells and the number of the LacZ positive cells were counted to calculate the effect of the gene transfection (number of LacZ positive cells/total number of cells (%)), and the average value was obtained. The results in the 2nd, 4th and 12th weeks are shown in FIG. 9.

FIG. 9 revealed that the highest level of expression was observed in the 2nd week and the medium level of expression was observed also in the 4th week but almost no expression was observed in the 12th week. This showed that the gene transfected into the periosteum was appropriately expressed in the initial to intermediate stage requiring the exhibition of its function and the expression was reduced in the later stage in which the cartilage was repaired and the exhibition of the function was no longer required (stage in which to exclude the expressed protein as a foreign matter).

The foregoing results proved that the gene could be transfected into the periosteum with the gene gun, and that the transfected gene could properly be expressed in the periosteal tissue in an appropriate stage in the periosteal tissue without involving the loss of the function, the inflammation and the disorder of the periosteal tissue.

5. Experiments of Transfection and Transplantation of HAS 2 Gene

Knees of 20 NZW rabbits (male, 3.0 kg) were grouped as follows.

(1) control group

2nd week: 10 knees 4th week: 10 knees

(2) HAS 2 transfection group (group with HAS 2 transfected)

2nd week: 10 knees 4th week: 10 knees

(Of 10 knees in each group, 5 knees were used for a tissue sample, and the other 5 knees for RT-PCR respectively).

With respect to both groups, in the above-mentioned manner, a defective portion of a joint cartilage was produced, and a periosteum was collected.

With respect to the control group, in the above-mentioned manner, gold particles (uncoated with genes) were introduced into the periosteum collected from each individual with the gene gun, and transplanted into the cartilage defective portion of each individual. The transplantation method is as described above.

With respect to the HAS 2 transfection group, the gold particles (coated with HAS 2) prepared in 2 were introduced into the periosteum collected from each individual with the gene gun in the above-mentioned manner. Then, the periosteum having the gene transfected therein was transplanted into the cartilage defective portion of each individual. The transplantation method is as described above. Further, in the 2nd and 4th weeks after the transplantation, the HE staining and the TB staining were conducted, and the histological evaluation was conducted in the above-mentioned manner.

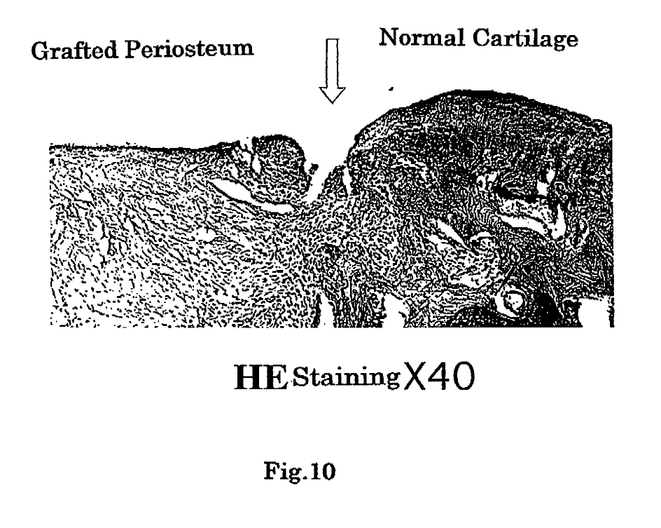

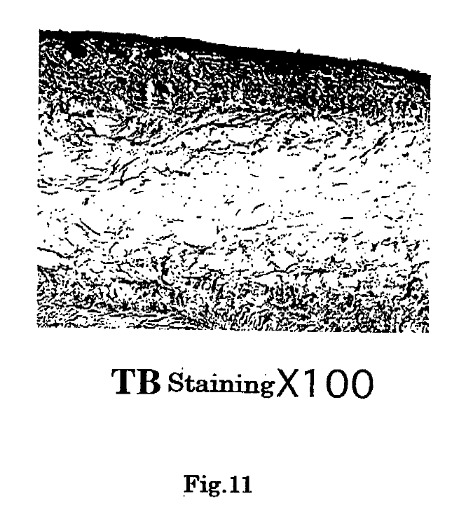

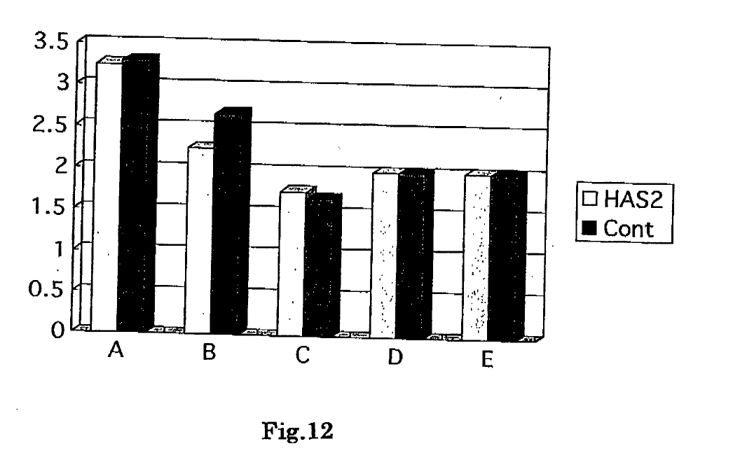

The typical HE stained image and TB stained image of the HAS 2 transfection group in the 2nd week are shown in FIGS. 10 and 11 respectively. Further, the histological evaluation of the control group and the HAS 2 transfection group in the 2nd week (averages of scores in items (a) to (e) in each group) is shown in FIG. 12.

FIGS. 10 to 12 revealed that in the 2nd week, the interface of the transplanted periosteum was discontinuous in both the control group and the HAS 2 transfection group, no metachromasia was exhibited in the toluidine blue staining and differentiation to a hyaline cartilage was not observed. With respect to the tissue repair scores, there was no difference with the control group in each item.

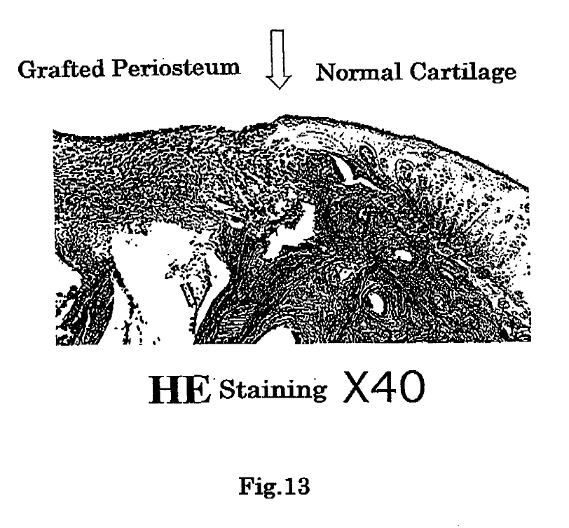

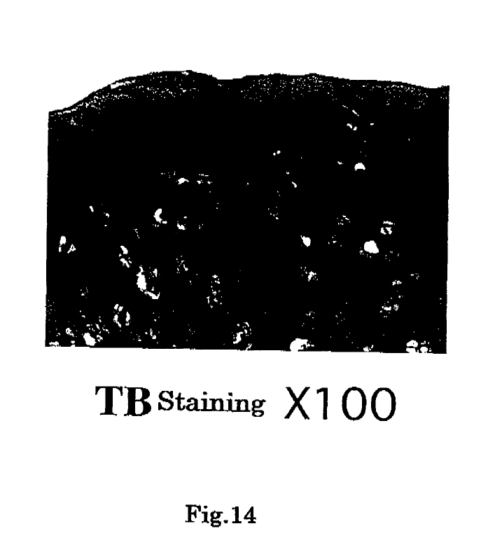

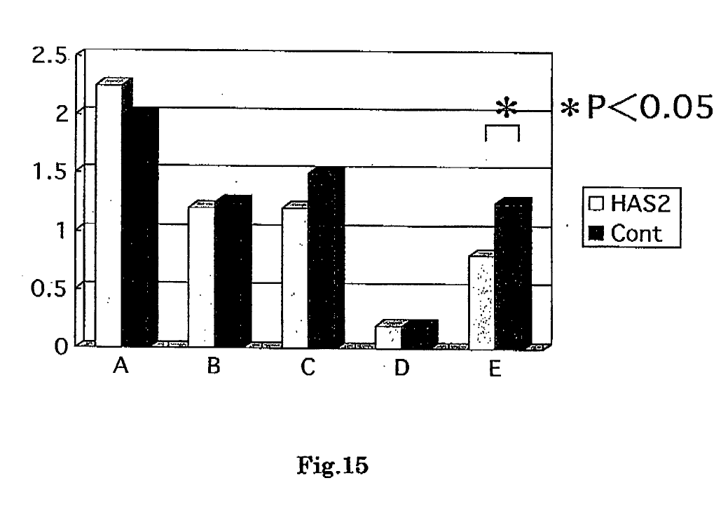

The typical HE stained image and TB stained image of the HAS 2 transfection group in the 4th week are shown in FIGS. 13 and 14 respectively. Further, the histological evaluation of the control group and the HAS 2 transfection group in the 4th week (averages of scores in items (a) to (e) in each group) is shown in FIG. 15.

FIGS. 13 to 15 revealed that in the 4th week, the good continuity of the interface was much observed in the HAS 2 transfection group. Further, in the toluidine blue staining, metachromasia was exhibited, showing that the group was being differentiated to a hyaline cartilage. With respect to the tissue repair scores, significantly good results were obtained in the HAS 2 transfection group on the “integration of a donor bony tissue in an adjacent portion to a host cartilaginous tissue”.

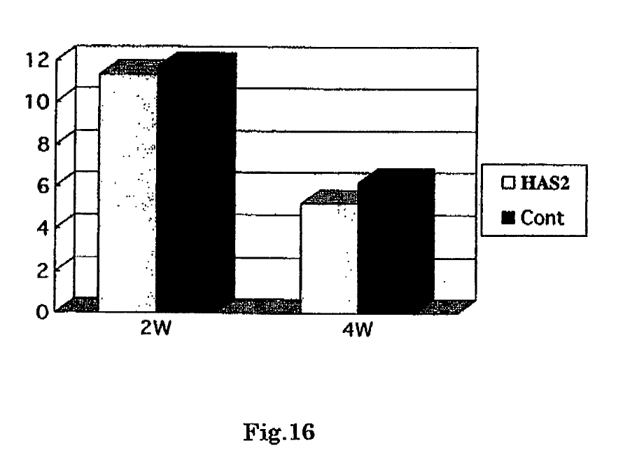

Histological evaluation of the control group and the HAS 2 transfection group in the 2nd and 4th weeks (averages of overall scores in each group) is shown in FIG. 16.

FIG. 16 revealed that the HAS 2 transfection group was histologically better in the tissue condition than the control group. This showed that when the HAS 2 gene was transfected into the periosteum with the gene gun and this periosteum was transplanted into the cartilage defective portion, the cartilage defective portion could be repaired to a nearly normal state in comparison with a case of not transfecting the gene.

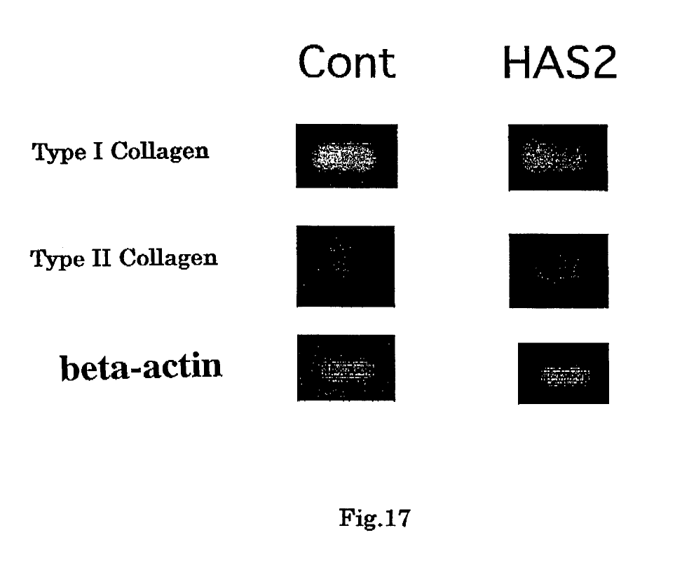

With respect to the control group and the HAS 2 transfection group, the expressions of I type collagen and II type collagen were analyzed using RT-PCR. The results in the 4th week are shown in FIG. 17. By the way, β-actin was used as a control.

FIG. 17 revealed that in the HAS 2 transfection group, the expression of type I collagen was slightly low and the expression of type II collagen was high in comparison with the control group. This showed that the use of the periosteum with the HAS 2 gene transfected through the gene gun was preferable because it expedited the synthesis of type II collagen as a main ingredient of the normal articular cartilage and it was differentiated to a tissue closer to the hyaline cartilage.

Separately from these experiments, an experiment (in vitro) was performed in which the HAS 2 genes were transfected into the culture chondrocytes in the above-mentioned manner and hyaluronic acid was detected using a hyaluronic acid binding protein (HABP). Consequently, it was identified that in the cells having the HAS 2 genes transfected therein, hyaluronic acid was produced in quite a large amount as compared with the cells free from the HAS 2 genes. From this experiment, it can be imagined that the HAS 2 gene transfected into the periosteum also exhibits HAS 2, the ability to synthesize hyaluronic acid is exhibited and hyaluronic acid is produced in a large amount.

The gene of the invention and the carrier of the invention are quite useful because these can be used for production of the carrier of the invention which carries the gene of the invention. The kit of the invention is quite useful because the production can be conducted more easily and quickly. The carrier of the invention which carries the gene of the invention is quite useful because it does not affect at all the bony tissue even though physically introduced in the bony tissue with the gene gun and the function of the bony tissue itself is not substantially impaired. When this bony tissue is transplanted into the cartilage defective portion, it is integrated as in the normal periosteum to form a cartilage. The gene transfected into the periosteum is properly expressed in an appropriate stage requiring the tissue repair, but the gene expression is reduced in a stage in which it is no longer necessary. This gene expression does not substantially affect the bony tissue. When a specific gene such as HAS 2 is transfected into the bony tissue and the resulting tissue is transplanted into the cartilage defective portion, the defective portion can be repaired well to a nearly normal state. Thus, it is quite useful. The invention tissue is quite useful because it dispenses with the transfection of the gene of the invention into the bony tissue, enabling the easier and quicker transplantation. The gene gun of the invention is quite useful because it can be used in the introduction of the carrier of the invention which carries the gene of the invention in the bony tissue and the production of the invention tissue.

Further, according to the invention, the gene can be transfected into the quite limited specific site of the in vivo bony tissue directly and instantaneously using the gene gun. Accordingly, quite easy and quick treatment is enabled as compared with a method in which a bony tissue is taken out and genes are then transfected therein over a fixed period of time. That is, the treatment can be completed by one operation without requiring plural operations. It has almost no adverse effect on the surrounding tissues that do not require the gene transfection.

In addition, since viral vectors are not used in the invention, a possibility of inducing inflammation or unnecessary constitutional symptoms can be eliminated. Thus, the invention is said to be quite useful.

What is claimed is:

1. A gene which is used for transfection into a bony tissue with a gene gun.

2. The gene according to claim 1, which is DNA encoding an enzyme protein.

3. The gene according to claim 2, wherein the DNA encoding the enzyme protein is DNA encoding a hyaluronic acid synthetase.

4. The gene according to claim 3, wherein the DNA encoding the hyaluronic acid synthetase is DNA encoding hyaluronic acid synthetase 2.

5. The gene according to claim 4, wherein the DNA encoding hyaluronic acid synthetase 2 is selected from the group consisiting of the following (a) to (c).
(a) DNA encoding a protein comprising an amino acid sequence represented by SEQ ID NO: 2.
(b) DNA encoding a protein comprising an amino acid sequence represented by SEQ ID NO: 2 in which one or several amino acids are deleted, substituted, inserted or transposed, the protein having a hyaluronic acid synthetase activity.
(c) DNA which is hybridizable with the DNA described in (a) or DNA complementary to the DNA described in (a), or DNA having a part of nucleotide sequences of these DNAs under stringent conditions.

6. The gene according to claim 5, which is DNA comprising a nucleotide sequence represented by Nucleotide Nos. 536 to 2191 in SEQ ID NO: 1.

7. The gene according to any one of claims 1 to 6, which is carried by a vector.

8. The gene according to claim 7, wherein the vector is an expression vector.

9. The gene according to any one of claims 1 to 8, wherein the bony tissue is a periosteal tissue or a cartilaginous tissue.

10. The gene according to claim 9, wherein the periosteal tissue or the cartilaginous tissue is to be grafted into a defective portion of an articular cartilage.

11. A carrier which is used for transfecting a gene into a bony tissue with a gene gun.

12. The carrier according to claim 11, which carries the gene according to any one of claims 1 to 10.

13. A kit comprising the gene according to any one of claims 1 to 10 and the carrier according to claim 11 as constituents.

14. A bony tissue transfected with the gene according to any one of claims 1 to 10.

15. A gene gun which is used for transfecting the gene according to any one of claims 1 to 10 into a bony tissue.

16. A method for transfecting a gene into a bony tissue, comprising transfecting the gene with a gene gun.

17. A method for treating cartilage defect, comprising the steps of:
a) transfecting a gene into a bony tissue with a gene gun; and
b) grafting the bony tissue obtained by the step a) into a defective protion of cartilage.

18. A method for repairing cartilage defect, comprising the steps of:
a) transfecting a gene into a bony tissue with a gene gun; and
b) grafting the bony tissue obtained by the step a) into a defective portion of cartilage.
